(19) United States
(12) Plant Patent Application Publication
(10) Pub. No.: US 2015/0020280 P1
(43) Pub. Date: Jan. 15, 2015

(54) Sutera plant named 'KLESG12346'

(50) ;

(71) Applicant: Klemm+Sohn GmbH & Co. KG, Stuttgart (DE)

(72) Inventors: Nils Klemm, Stuttgart (DE); Katinka Wilde, Stuttgart (DE)

(21) Appl. No.: 13/987,184

(22) Filed: Jul. 9, 2013

(51) Int. Cl. A01H 5/00

(52) U.S. Cl. PLT/485

(57) ABSTRACT

A new Sutera plant particularly distinguished by having dark blue flowers, a trailing plant habit, and good branching, is disclosed.

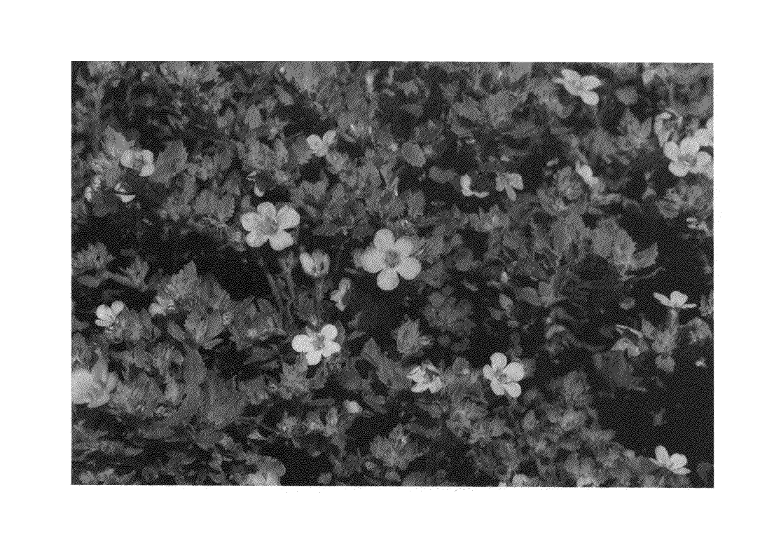

### GENUS AND SPECIES

Sutera grandiflora

### VARIETY DENOMINATION

‘KLESG12346’

### BACKGROUND OF THE NEW PLANT

The present invention comprises a new and distinct variety of Sutera, botanically known as Sutera grandiflora, and hereinafter referred to by the variety name ‘KLESG12346’. ‘KLESG12346’ originated from an open-pollination between the proprietary female Sutera variety ‘SnBigPinki’ (unpatented) and an unknown male Sutera variety in the summer of 2009.

In August of 2009, seed was collected from the open-pollination between the two Sutera parental lines, ‘SnBigPinki’ and an unknown Sutera in Stuttgart, Germany. The seeds were sown and plants were grown for evaluation. A plant line was selected in May 2010 which had dark blue flowers and was subsequently named ‘KLESG12346’. In June 2010, ‘KLESG12346’ was first vegetatively propagated by in-vitro cultivation. ‘KLESG12346’ was found to reproduce true to type in successive generations of asexual propagation via in-vitro cultivation.

### SUMMARY

The following are the most outstanding and distinguishing characteristics of this new variety when grown under normal horticultural practices in Stuttgart, Germany.

1. Dark blue flower color;

2. A trailing plant habit; and

3. Good branching.

### DESCRIPTION OF THE PHOTOGRAPH

This new Sutera plant is illustrated by the accompanying photograph which shows the foliage, buds, and a close-up of the flowers. The photograph is of five-month-old plant grown in Stuttgart, Germany in August 2011. The colors shown are as true as can be reasonably obtained by conventional photographic procedures.

### DESCRIPTION OF THE NEW VARIETY

The following detailed descriptions set forth the distinctive characteristics of ‘KLESG12346’. The data which define these characteristics were collected from asexual reproductions carried out in Stuttgart, Germany. Data was collected on 11-week-old plants grown in a controlled greenhouse environment in Stuttgart, Germany in May 2013. Color references are to The R.H.S. Colour Chart of The Royal Horticultural Society of London (R.H.S.) (2005).
Classification:
   Family.—Scrophulariaceae.
   Botanical.—Sutera grandiflora.
   Common.—Sutera.
   Designation.—‘KLESG12346’.
Parentage:
   Female parent.—Proprietary Sutera line ‘SnBigPinki’ (unpatented).
   Male parent.—An unknown Sutera line.
Plant:
   Habit.—Trailing.
   Height.—About 10.0 cm.
   Spread.—33.0 cm.
   Life cycle.—Blooms from April until September.
   Form.—Round.
   Time to produce a rooted cutting.—4 weeks.
   Time to bloom from propagation.—5 weeks.
   Lastingness of inflorescence on the plant.—About 7 days.
Stems:
   Number of stems per plant.—6.
   Length.—About 20.0 cm.
   Diameter.—0.5 cm.
   Internode length.—About 1.5 cm.
   Color.—RHS 144A.
   Texture.—Smooth.
   Anthocyanin.—Absent.
Leaves:
   Arrangement.—Single, opposite.
   Shape.—Ovate, acute angle to the stem.
   Apex.—Acute.
   Base.—Cordate.
   Margin.—Serrate.
   Length.—Between 1.1 cm and 2.0 cm.
   Width.—Between 1.1 cm and 2.4 cm.
   Color (both immature and mature leaves).—Upper surface: RHS 137A Lower surface: RHS 137C.
   Appearance (both upper and lower surfaces).—Leathery.
   Texture (both upper and lower surfaces).—Leathery.
   Venation type.—Pinnate.
   Venation color (both upper and lower surfaces).—RHS 144A.
   Fragrance.—Present.
Inflorescence:
   Blooming habit.—Freely flowering.
   Quantity of inflorescence per stem.—About 9.
   Type of inflorescence.—Flowers are often solitary, most often 2 flowers per node are developed.
   Fragrance.—Present.
   Pedicel.—Color: RHS 144A Length: About 0.8 cm to 1.0 cm Diameter: About 0.1 cm Texture: Pubescent.
Bud:
   Shape.—Tubular.
   Diameter.—0.4 cm. Length: 0.3 cm Color (at tight bud): RHS 155A.
Corolla (flower):
   Corolla type and shape.—Single-type, nearly round, lower part tube and funnel shaped formed by 5 fused petals, upper part is salver shaped formed by the partly free petal lobes.
   Corolla, horizontal diameter.—1.8 cm.
   Corolla, total length.—1.6 cm.
   Throat.—Color, inner surface: RHS 23A Color, outer surface: RHS 15B Diameter: 0.4 cm Texture of throat (both inner and outer surfaces): Smooth.
Petal (lobes):
   Apex.—Rounded tips.
   Base.—Fused.
   Margin.—Entire.
   Color.—Upper surface: RHS 93D Lower surface: RHS 90B.
   Texture (both upper and lower surfaces).—Smooth.
Calyx:
   Arrangement and appearance.—Whorl of 5 sepals surrounding the base of the flower tube, sepals are fused at the base.
   Sepals.—Color (both upper and lower surfaces): RHS 141D Length: 1.1 cm Width: 0.1 cm Apex: Acute Base: Fused Margin: Entire Texture (both upper and lower surfaces): Densely pubescent.
Reproductive organs:
   Stamens.—Quantity: 4 Filament: Color: RHS 155B Length: 0.6 cm Diameter: 0.1 cm Anther color: RHS 15D Pollen amount: Abundant Pollen color: RHS 22A.
   Pistil.—Quantity per flower: 1 Length: 1.1 cm Stigma color: RHS 145C Style color: RHS 155B.
Fruit and seed set:
   Capsulates/fruit.—Color: RHS 137C Size: 0.5 cm×0.4 cm Shape: Ovate.
   Seeds.—Color: RHS 20A Size: About 0.1 cm Shape: Round.
Disease and pest/insect resistance: None observed.

### COMPARISON WITH PARENTAL LINE AND KNOWN VARIETY

‘KLESG12346’ is distinguished from its female parent as described in Table 1:

TABLE 1
Comparison with Parental Line

| Trait | ‘KLESG12346’ | ‘SnBigPinki’ |
|---|---|---|
| Flower color | Dark blue | Pink |

‘KLESG12346’ is most similar to the Sutera plant named ‘Gulliver Lavender’ (patent status unknown). Differences between the two varieties are described in Table 2:

TABLE 2
Comparison with Similar Variety

| Trait | ‘KLESG12346’ | ‘Gulliver Lavender’ |
|---|---|---|
| Flower color | Dark blue | Light blue |
| Branching | More branching than | Less branching than |
| | ‘Gulliver Lavender’ | ‘KLESG12346’ |

We claim:

1. A new and distinct variety of Sutera plant named ‘KLESG12346’ as shown and described herein.
